(19) United States
(12) Patent Application Publication
(10) Pub. No.: US 2026/0086633 A1
(43) Pub. Date: Mar. 26, 2026

(54) PUPIL REACTIVITY TESTING

(71) Applicant: Apple Inc., Cupertino, CA (US)

(72) Inventors: Jay C. MUNG, San Jose, CA (US); Alexander G. BERARDINO, San Francisco, CA (US); Andrew B. WATSON, Los Gatos, CA (US); Ankit GUPTA, Santa Clara, CA (US); Anselm GRUNDHOEFER, Campbell, CA (US); Anshuman MISHRA, Sunnyvale, CA (US); Harihar NARASIMHA-IYER, Sunnyvale, CA (US); Jeremy T. WARSHAUER, Burlingame, CA (US); Lyndon DUONG, San Francisco, CA (US); Mehmet N. AGAOGLU, Dublin, CA (US); Peng LIU, San Jose, CA (US)

(21) Appl. No.: 19/335,814

(22) Filed: Sep. 22, 2025

Related U.S. Application Data
(60) Provisional application No. 63/699,122, filed on Sep. 25, 2024.

(51) Int. Cl. G06F 3/01; A61B 3/11

(52) U.S. Cl. CPC G06F 3/013; A61B 3/112

(57) ABSTRACT

The present disclosure is generally related to systems and methods for generating an indication of a pupillary response deviation. An electronic device in communication with a display and one or more input devices, displays one or more images. Further, the method includes, detecting, one or more first sizes of a pupil of a user. In response to detecting the one or more first sizes of a pupil, the one or more first sizes of the pupil are compared with one or more second sizes of the pupil of the user. Moreover, in accordance with a determination that one or more criteria are satisfied, including a criterion that is satisfied when the one or more first sizes of the pupil deviate from the one or more second sizes of the pupil by one or more pupil size thresholds, generate an indication of a deviation from the expected pupillary response.

### CROSS REFERENCE TO RELATED APPLICATIONS

This application claims the benefit of U.S. Provisional Application No. 63/699,122, filed Sep. 25, 2024, the content of which is herein incorporated by reference in its entirety for all purposes.

### FIELD OF THE DISCLOSURE

This relates generally to systems and methods for measuring ocular parameters based on a change in content being displayed on an electronic device in a computer-generated environment.

### BACKGROUND OF THE DISCLOSURE

The development of monitoring ocular parameters has increased in recent years. Example health monitoring includes a head mounted display. Input devices, such as cameras, controllers, joysticks, touch-sensitive surfaces, and touch-screen displays for electronic devices and other electronic computing devices are used to interact with head mounted displays.

### SUMMARY OF THE DISCLOSURE

This relates generally to systems and methods for measuring ocular parameters based on a change in content being displayed on an electronic device in a computer-generated environment. Some examples of the disclosure are directed to systems and methods for generating an indication of a pupillary response deviation. In some examples, an electronic device in communication with a display and one or more input devices displays, via the one or more displays, one or more images. In some examples, while displaying the one or more images, the electronic device detects, via the one or more input devices, one or more first sizes of a pupil of a user of an electronic device. In some examples, in response to detecting the one or more first sizes of a pupil, comparing the one or more first sizes of the pupil detected, while displaying the one or more images, with one or more second sizes of the pupil of the user of the electronic device, the one or more images. In some examples, in accordance with a determination that one or more criteria are satisfied, including a criterion that is satisfied when the one or more first sizes of the pupil deviate from the one or more second sizes of the pupil by one or more pupil size thresholds, generate an indication of a deviation from the expected pupillary response.

The full descriptions of these examples are provided in the Drawings and the Detailed Description, and it is understood that this Summary does not limit the scope of the disclosure in any way.

### BRIEF DESCRIPTION OF THE DRAWINGS

For improved understanding of the various examples described herein, reference should be made to the Detailed Description below along with the following drawings. Like reference numerals often refer to corresponding parts throughout the drawings.

FIG. 1 illustrates an electronic device presenting an extended reality environment according to some examples of the disclosure.

FIG. 2 illustrates a block diagram of an example architecture for a device according to some examples of the disclosure.

FIGS. 3A-3C illustrate an electronic device with a display having varying brightness levels according to some examples of the disclosure.

FIG. 3D illustrates a graph showing a relationship between power draw/luminance over time with respective expected pupil sizes according to some examples of the disclosure

FIGS. 4A-4B illustrate an electronic device with varying environment passthrough levels according to some examples of the disclosure.

FIGS. 5A-5B illustrate a graph showing a comparison between measured pupil size and expected pupil size according to some examples of the disclosure.

FIGS. 6A-6B illustrate an exemplary display of an electronic according to some examples of the disclosure.

FIG. 7 is a flow diagram illustrating an example process for generating an indication of a deviation from an expected pupillary response according to some examples of the disclosure.

FIG. 8 is a flow diagram illustrating an example process for determining whether to opportunistically measure pupil sizes of one or more eyes of a user of an electronic device according to some examples of the disclosure.

### DETAILED DESCRIPTION

Some examples of the disclosure are directed to systems and methods for generating an indication of a pupillary response deviation. In some examples, an electronic device in communication with a display and one or more input devices, displays, via the one or more displays, one or more images. In some examples, while displaying the one or more images, detecting, via the one or more input devices, one or more first sizes of a pupil of a user of an electronic device. In some examples, in response to detecting the one or more first sizes of a pupil, comparing the one or more first sizes of the pupil detected, while displaying the one or more images, with one or more second sizes of the pupil of the user of the electronic device, the one or more images. In some examples, in accordance with a determination that one or more criteria are satisfied, including a criterion that is satisfied when the one or more first sizes of the pupil deviate from the one or more second sizes of the pupil by one or more pupil size thresholds, generate an indication of a deviation from the expected pupillary response.

As used herein, an object that is displayed in a head-locked orientation in a three-dimensional environment has a distance and orientation offset relative to the user's head. In some examples, a head-locked object moves within the three-dimensional environment as the user's head moves (as the viewpoint of the user changes).

As used herein, an object that is displayed in a world-locked orientation in a three-dimensional environment does not have a distance or orientation offset relative to the user.

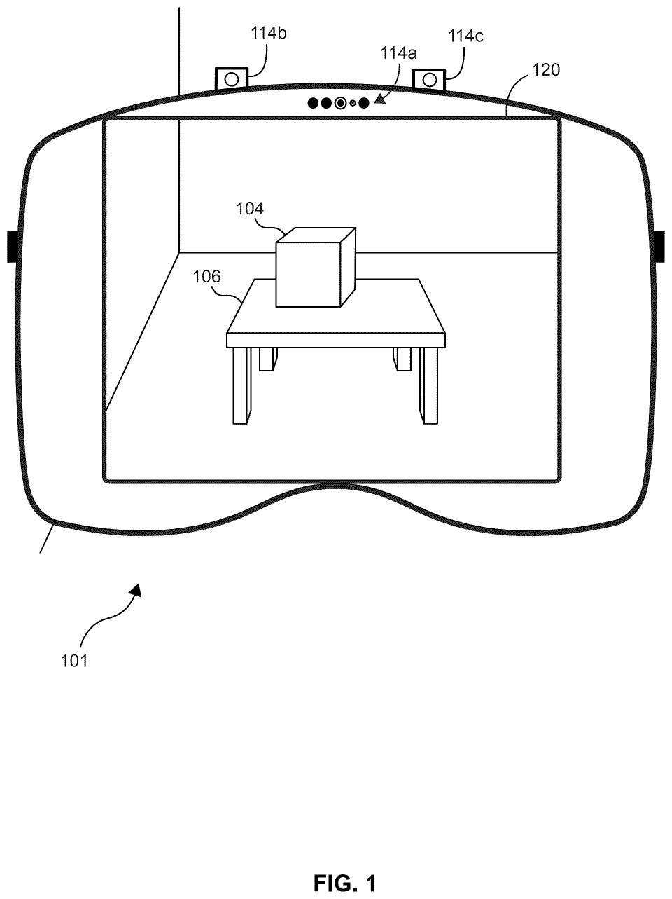

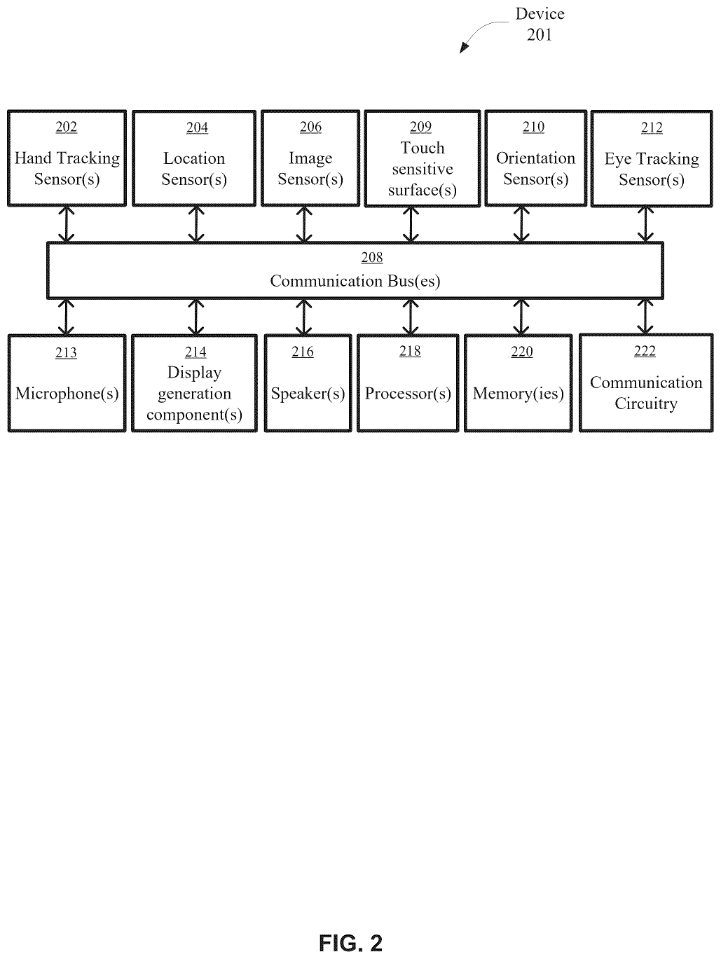

FIG. 1 illustrates an electronic device 101 presenting an extended reality (XR) environment (e.g., a computer-generated environment optionally including representations of physical and/or virtual objects) according to some examples of the disclosure. In some examples, as shown in FIG. 1, electronic device 101 is a head-mounted display or other head-mountable device configured to be worn on a head of a user of the electronic device 101. Examples of electronic device 101 are described below with reference to the architecture block diagrams of FIG. 2. As shown in FIG. 1, electronic device 101 and table 106 are located in a physical environment. The physical environment may include physical features such as a physical surface (e.g., floor, walls) or a physical object (e.g., table, lamp, etc.). In some examples, electronic device 101 may be configured to detect and/or capture images of physical environment including table 106 (illustrated in the field of view of electronic device 101).

In some examples, as shown in FIG. 1, electronic device 101 includes one or more internal image sensors 114a oriented towards a face of the user (e.g., eye tracking cameras described below with reference to FIG. 2). In some examples, internal image sensors 114a are used for eye tracking (e.g., detecting a gaze of the user). Internal image sensors 114a are optionally arranged on the left and right portions of display 120 to enable eye tracking of the user's left and right eyes. In some examples, electronic device 101 also includes external image sensors 114b and 114c facing outwards from the user to detect and/or capture the physical environment of the electronic device 101 and/or movements of the user's hands or other body parts.

In some examples, display 120 has a field of view visible to the user (e.g., that may or may not correspond to a field of view of external image sensors 114b and 114c). Because display 120 is optionally part of a head-mounted device, the field of view of display 120 is optionally the same as or similar to the field of view of the user's eyes. In other examples, the field of view of display 120 may be smaller than the field of view of the user's eyes. In some examples, electronic device 101 may be an ocular see-through device in which display 120 is a transparent or translucent display through which portions of the physical environment may be directly viewed. In some examples, display 120 may be included within a transparent lens and may overlap all or only a portion of the transparent lens. In other examples, electronic device may be a video-passthrough device in which display 120 is an opaque display configured to display images of the physical environment captured by external image sensors 114b and 114c. While a single display 120 is shown, it should be appreciated that display 120 may include a stereo pair of displays.

In some examples, in response to a trigger, the electronic device 101 may be configured to display a virtual object 104 in the XR environment represented by a cube illustrated in FIG. 1, which is not present in the physical environment, but is displayed in the XR environment positioned on the top of real-world table 106 (or a representation thereof). Optionally, virtual object 104 can be displayed on the surface of the table 106 in the XR environment displayed via the display 120 of the electronic device 101 in response to detecting the planar surface of table 106 in the physical environment 100.

It should be understood that virtual object 104 is a representative virtual object and one or more different virtual objects (e.g., of various dimensionality such as two-dimensional or other three-dimensional virtual objects) can be included and rendered in a three-dimensional XR environment. For example, the virtual object can represent an application or a user interface displayed in the XR environment. In some examples, the virtual object can represent content corresponding to the application and/or displayed via the user interface in the XR environment. In some examples, the virtual object 104 is optionally configured to be interactive and responsive to user input (e.g., air gestures, such as air pinch gestures, air tap gestures, and/or air touch gestures), such that a user may virtually touch, tap, move, rotate, or otherwise interact with, the virtual object 104.

In some examples, displaying an object in a three-dimensional environment may include interaction with one or more user interface objects in the three-dimensional environment. For example, initiation of display of the object in the three-dimensional environment can include interaction with one or more virtual options/affordances displayed in the three-dimensional environment. In some examples, a user's gaze may be tracked by the electronic device as an input for identifying one or more virtual options/affordances targeted for selection when initiating display of an object in the three-dimensional environment. For example, gaze can be used to identify one or more virtual options/affordances targeted for selection using another selection input. In some examples, a virtual option/affordance may be selected using hand-tracking input detected via an input device in communication with the electronic device. In some examples, objects displayed in the three-dimensional environment may be moved and/or reoriented in the three-dimensional environment in accordance with movement input detected via the input device.

In the discussion that follows, an electronic device that is in communication with a display generation component and one or more input devices is described. It should be understood that the electronic device optionally is in communication with one or more other physical user-interface devices, such as a touch-sensitive surface, a physical keyboard, a mouse, a joystick, a hand tracking device, an eye tracking device, a stylus, etc. Further, as described above, it should be understood that the described electronic device, display and touch-sensitive surface are optionally distributed amongst two or more devices. Therefore, as used in this disclosure, information displayed on the electronic device or by the electronic device is optionally used to describe information outputted by the electronic device for display on a separate display device (touch-sensitive or not). Similarly, as used in this disclosure, input received on the electronic device (e.g., touch input received on a touch-sensitive surface of the electronic device, or touch input received on the surface of a stylus) is optionally used to describe input received on a separate input device, from which the electronic device receives input information.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, a television channel browsing application, and/or a digital video player application.

FIG. 2 illustrates a block diagram of an example architecture for an electronic device 201 according to some examples of the disclosure. In some examples, electronic device 201 includes one or more electronic devices. For example, the electronic device 201 may be a portable device, an auxiliary device in communication with another device, a head-mounted display, etc., respectively. In some examples, electronic device 201 corresponds to electronic device 101 described above with reference to FIG. 1.

As illustrated in FIG. 2, the electronic device 201 optionally includes various sensors, such as one or more hand tracking sensors 202, one or more location sensors 204, one or more image sensors 206 (optionally corresponding to internal image sensors 114a and/or external image sensors 114b and 114c in FIG. 1), one or more touch-sensitive surfaces 209, one or more motion and/or orientation sensors 210, one or more eye tracking sensors 212, one or more microphones 213 or other audio sensors, one or more body tracking sensors (e.g., torso and/or head tracking sensors), one or more display generation components 214, optionally corresponding to display 120 in FIG. 1, one or more speakers 216, one or more processors 218, one or more memories 220, and/or communication circuitry 222. One or more communication buses 208 are optionally used for communication between the above-mentioned components of electronic devices 201.

Communication circuitry 222 optionally includes circuitry for communicating with electronic devices, networks, such as the Internet, intranets, a wired network and/or a wireless network, cellular networks, and wireless local area networks (LANs). Communication circuitry 222 optionally includes circuitry for communicating using near-field communication (NFC) and/or short-range communication, such as Bluetooth®.

Processor(s) 218 include one or more general processors, one or more graphics processors, and/or one or more digital signal processors. In some examples, memory 220 is a non-transitory computer-readable storage medium (e.g., flash memory, random access memory, or other volatile or non-volatile memory or storage) that stores computer-readable instructions configured to be executed by processor(s) 218 to perform the techniques, processes, and/or methods described below. In some examples, memory 220 can include more than one non-transitory computer-readable storage medium. A non-transitory computer-readable storage medium can be any medium (e.g., excluding a signal) that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, ocular, and/or semiconductor storages. Examples of such storage include magnetic disks, ocular discs based on compact disc (CD), digital versatile disc (DVD), or Blu-ray technologies, as well as persistent solid-state memory such as flash, solid-state drives, and the like.

In some examples, display generation component(s) 214 include a single display (e.g., a liquid-crystal display (LCD), organic light-emitting diode (OLED), or other types of display). In some examples, display generation component(s) 214 includes multiple displays. In some examples, display generation component(s) 214 can include a display with touch capability (e.g., a touch screen), a projector, a holographic projector, a retinal projector, a transparent or translucent display, etc. In some examples, electronic device 201 includes touch-sensitive surface(s) 209, respectively, for receiving user inputs, such as tap inputs and swipe inputs or other gestures. In some examples, display generation component(s) 214 and touch-sensitive surface(s) 209 form touch-sensitive display(s) (e.g., a touch screen integrated with electronic device 201 or external to electronic device 201 that is in communication with electronic device 201).

Electronic device 201 optionally includes image sensor(s) 206. Image sensors(s) 206 optionally include one or more visible light image sensors, such as charged coupled device (CCD) sensors, and/or complementary metal-oxide-semiconductor (CMOS) sensors operable to obtain images of physical objects from the real-world environment. Image sensor(s) 206 also optionally include one or more infrared (IR) sensors, such as a passive or an active IR sensor, for detecting infrared light from the real-world environment. For example, an active IR sensor includes an IR emitter for emitting infrared light into the real-world environment. Image sensor(s) 206 also optionally include one or more cameras configured to capture movement of physical objects in the real-world environment. Image sensor(s) 206 also optionally include one or more depth sensors configured to detect the distance of physical objects from electronic device 201. In some examples, information from one or more depth sensors can allow the device to identify and differentiate objects in the real-world environment from other objects in the real-world environment. In some examples, one or more depth sensors can allow the device to determine the texture and/or topography of objects in the real-world environment.

In some examples, electronic device 201 uses CCD sensors, event cameras, and depth sensors in combination to detect the physical environment around electronic device 201. In some examples, image sensor(s) 206 include a first image sensor and a second image sensor. The first image sensor and the second image sensor work in tandem and are optionally configured to capture different information of physical objects in the real-world environment. In some examples, the first image sensor is a visible light image sensor and the second image sensor is a depth sensor. In some examples, electronic device 201 uses image sensor(s) 206 to detect the position and orientation of electronic device 201 and/or display generation component(s) 214 in the real-world environment. For example, electronic device 201 uses image sensor(s) 206 to track the position and orientation of display generation component(s) 214 relative to one or more fixed objects in the real-world environment.

In some examples, electronic device 201 includes microphone(s) 213 or other audio sensors. Electronic device 201 optionally uses microphone(s) 213 to detect sound from the user and/or the real-world environment of the user. In some examples, microphone(s) 213 includes an array of microphones (a plurality of microphones) that optionally operate in tandem, such as to identify ambient noise or to locate the source of sound in space of the real-world environment.

Electronic device 201 includes location sensor(s) 204 for detecting a location of electronic device 201 and/or display generation component(s) 214. For example, location sensor(s) 204 can include a global position system (GPS) receiver that receives data from one or more satellites and allows electronic device 201 to determine the device's absolute position in the physical world.

Electronic device 201 includes orientation sensor(s) 210 for detecting orientation and/or movement of electronic device 201 and/or display generation component(s) 214. For example, electronic device 201 uses orientation sensor(s) 210 to track changes in the position and/or orientation of electronic device 201 and/or display generation component(s) 214, such as with respect to physical objects in the real-world environment. Orientation sensor(s) 210 optionally include one or more gyroscopes and/or one or more accelerometers.

Electronic device 201 includes hand tracking sensor(s) 202 and/or eye tracking sensor(s) 212 (and/or other body tracking sensor(s), such as leg, torso and/or head tracking sensor(s)), in some examples. Hand tracking sensor(s) 202 are configured to track the position/location of one or more portions of the user's hands, and/or motions of one or more portions of the user's hands with respect to the extended reality environment, relative to the display generation component(s) 214, and/or relative to another defined coordinate system. Eye tracking sensor(s) 212 are configured to track the position and movement of a user's gaze (eyes, face, or head, more generally) with respect to the real-world or extended reality environment and/or relative to the display generation component(s) 214. In some examples, hand tracking sensor(s) 202 and/or eye tracking sensor(s) 212 are implemented together with the display generation component(s) 214. In some examples, the hand tracking sensor(s) 202 and/or eye tracking sensor(s) 212 are implemented separate from the display generation component(s) 214.

In some examples, the hand tracking sensor(s) 202 (and/or other body tracking sensor(s), such as leg, torso and/or head tracking sensor(s)) can use image sensor(s) 206 (e.g., one or more IR cameras, 3D cameras, depth cameras, etc.) that capture three-dimensional information from the real-world including one or more body parts (e.g., hands, legs, or torso of a human user). In some examples, the hands can be resolved with sufficient resolution to distinguish fingers and their respective positions. In some examples, one or more image sensors 206 are positioned relative to the user to define a field of view of the image sensor(s) 206 and an interaction space in which finger/hand position, orientation and/or movement captured by the image sensors are used as inputs (e.g., to distinguish from a user's resting hand or other hands of other persons in the real-world environment). Tracking the fingers/hands for input (e.g., gestures, touch, tap, etc.) can be advantageous in that it does not require the user to touch, hold or wear any sort of beacon, sensor, or other marker.

In some examples, eye tracking sensor(s) 212 includes at least one eye tracking camera (e.g., infrared (IR) cameras) and/or illumination sources (e.g., IR light sources, such as LEDs) that emit light towards a user's eyes. The eye tracking cameras may be pointed towards a user's eyes to receive reflected IR light from the light sources directly or indirectly from the eyes. In some examples, both eyes are tracked separately by respective eye tracking cameras and illumination sources, and a focus/gaze can be determined from tracking both eyes. In some examples, one eye (e.g., a dominant eye) is tracked by one or more respective eye tracking cameras/illumination sources.

Electronic device 201 is not limited to the components and configuration of FIG. 2, but can include fewer, other, or additional components in multiple configurations. In some examples, electronic device 201 can be implemented between two electronic devices (e.g., as a system). In some such examples, each of (or more) electronic device may each include one or more of the same components discussed above, such as various sensors, one or more display generation components, one or more speakers, one or more processors, one or more memories, and/or communication circuitry. A person or persons using electronic device 201, is optionally referred to herein as a user or users of the device.

Attention is now directed towards examples of monitoring one or more ocular parameters. For example, an electronic device 301 (e.g., electronic device 201) may be used to monitor one or more ocular parameters. In some examples, one or more ocular parameters may include pupil size, iris size, or things of the like. Pupil size may be indicated using a pupil diameter, a pupil radius, a pupil circumference, volume, or any suitable measurement. Pupil size may be dependent on an amount of light being exposed to an eye. As more light is exposed to an eye, the smaller the pupil size is. Alternatively, as less light is exposed to an eye, the larger the pupil size is. This is due to the iris of the eye contracting when there is more light exposed to the eye, and relaxing when there is less light exposed to the eye. In some examples, an iris size may be indicated using an amount of visible iris. For example, the amount of visible iris may increase when more light is exposed to the eye, while the amount of visible iris may decrease when there is less light exposed to the eye. Because of the eye's natural reactivity to light exposure, there is an opportunity to monitor one or more ocular parameters of a user's eye while displaying one or more images on electronic device 301. Additionally, or alternatively, metrics such as resting pupil diameter, amplitude, latency, duration, velocity of pupil contraction and dilation may be measured.

Electronic device 301 may be used as an alternative to traditional methods for pupillary measurements. Traditional methods may include clinical visits and/or specialized pupillometer apparatuses. Advantageously, systems and methods described herein may serve as a more convenient alternative. For example, the electronic device 301 may display, via a display 320, one or more images with varying luminance. It should be noted that display 320 is exemplary, and electronic device 301 may include one or more displays. In some examples, display 320 may include one or more images with distinct changes in brightness (e.g., dark-to-light and light-to-dark). Advantageously, obtaining pupillary measurements while displaying one or more images using display 320 does not require a clinical setting and/or a specialized apparatus to evaluate one or more eyes (e.g., one or both eyes) of a user of the electronic device 301.

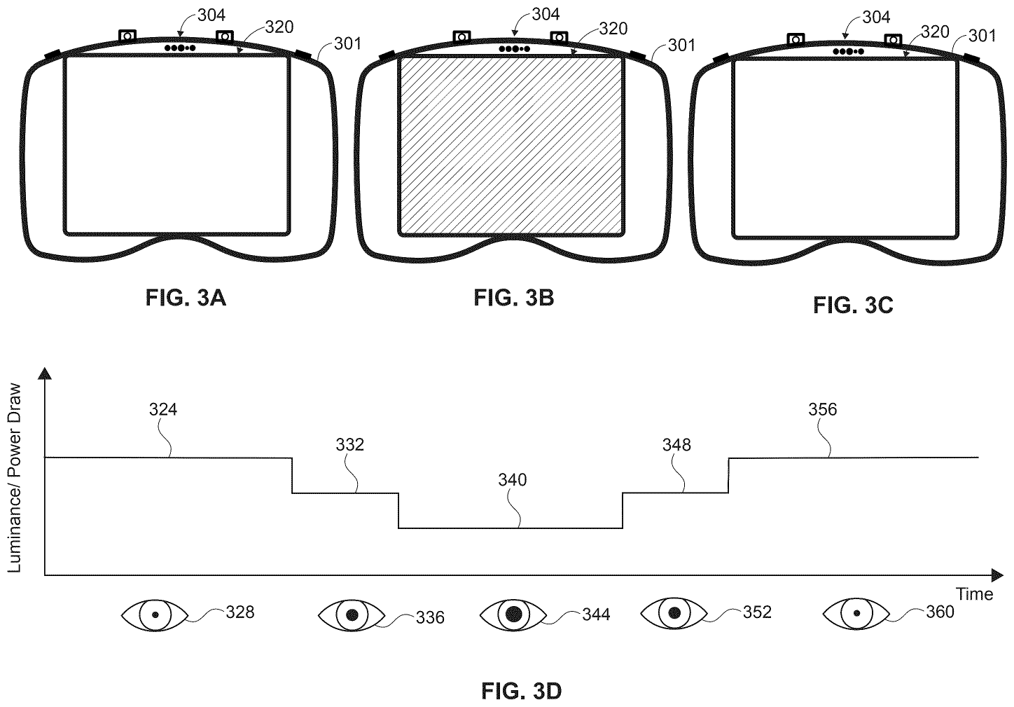

As shown in FIGS. 3A-3C, electronic device 301 may be a head mountable system/device and/or projection-based system/device (including a hologram-based system/device), such as, for example, heads-up displays (HUDs), head mounted displays (HMDs), windows having integrated display capability, displays formed as lenses designed to be placed on a person's eyes (e.g., similar to contact lenses), respectively. In the examples of FIGS. 3A-3C, electronic device 301 may be a head mounted display configured to display one or more images (e.g., digital content) on the display 320. In some examples, electronic device 301 may include one or more cameras 304. The one or more cameras 304 of the electronic device 301 may obtain pupillary measurements routinely. That is, after a predetermined time interval (e.g., each second, each minute, each day, each week) the one or more cameras 304 may obtain pupillary measurements. It should be noted that the frequency of measurements may change after a first measurement. For example, the predetermined time interval may increase from each minute to each day after a first measurement is obtained. Further, the one or more cameras 304 may have eye-tracking capabilities that allow the one or more cameras 304 to capture high fidelity measurements of a pupil size of the one or more eyes of the user of the electronic device 301. In combination with having known amounts of light that are being emitted to the user's eyes from display 320, electronic device 301 may be able to obtain pupillary measurements, and further, make determinations pertaining to the user's eye described in more detail below, without necessitating a specialized apparatus. For example, the known amounts of light being emitted to the user's eye are directly correlated to a power draw of the electronic device 301. As in, a larger amount of light being emitted (e.g., a bright image) requires more power draw from the electronic device 301. On the other hand, the amount of light being emitted to the user's eye is inversely related to the pupil size of the one or more eyes of the user of the electronic device 301. That is, when there is a larger amount of light being emitted (e.g., a bright image) to the user's eye, the respective pupils of the one or more eyes of the user of the electronic device 301 may decrease in size. As such, a user of the electronic device 301 may be able to track their eye measurements more efficiently.

Still referring to FIGS. 3A-3C, the display 320 may present one or more images, each of which having one or more image characteristics. In some examples, the one or more image characteristics may include, but are not limited to, total light values, luminance data (e.g., luminance values) brightness, or the like. In some examples, the luminance data may provide the display 320 with instructions pertaining to color and brightness that needs to be applied to each pixel of the display, depending on the image. In some examples, the luminance values may correspond to or be the same as display data that the electronic device 301 uses as instructions for displaying images. That is, the luminance values may be provided to the electronic device 301 rather than being measured by the electronic device 301. The luminance values may be represented by an average (e.g., a weight average) of luminance values in a portion of the display 320. In some examples, luminance values may be represented by extrema luminance values (minimum, maximum). For example, an area of the display 320 may have a local maximum luminance value, and that value may be used to represent the luminance value of the area. Additionally, or alternatively, a local minimum value may be used to represent the luminance value of the area on the display. In some examples, the luminance values may be aggregated to account for unevenness in illumination of the one or more images. For example, the display 320 may display luminance values in area of a gaze direction of the user of the electronic device (e.g., perimeter 608). The luminance values that are displayed in the gaze direction may be aggregated (e.g., summed) to yield an aggregate luminance value for the area of a gaze direction.

In some examples, the one or more image characteristics may be correlated to a power draw of the display 320. For example, one or more images may have a high luminance value, and cause the display 320 to draw more power from a power source of the electronic device 301 than the power draw needed to display one or more images with a lower luminance value. In some examples, power draw by the display 320 may be monitored by the electronic device 301. For example, the power draw required to display one or more images using display 320 may be measured at a value above a predetermined threshold. The predetermined power draw threshold associated with the display 320 may be set based at least on an amount of heat generated by the electronic device 301 as the power draw increases. For example, as the power draw from display 320 increases, an amount of heat generated by the electronic device 301 may increase. However, the functionality of the electronic device 301 may be limited when the electronic device 301 generates too much heat. Accordingly, the electronic device 301 may restrict the display's 320 power draw (e.g., set a power draw threshold) and limit functionality similarly to a low power mode, as described in further detail below, to control the amount of heat the electronic device 301 generates. It should be noted that the electronic device 301 may limit the power draw of the display 320 to preserve electrical components of the electrical device 301 and ensure the safety of the user of the electronic device. In accordance with a determination that the power draw required to display one or more images using display 320 exceeds the predetermined threshold, the electronic device 301 may forgo displaying the version of the one or more images that would require the power draw above the predefined threshold and display a modified version of the one or more images. The modified version may include one or more images with adjusted (e.g., dimmed) image characteristics such that the power draw required by the display 320 is below the predetermined threshold.

In some examples, the electronic device 301 may be in a “low power” mode. The low power mode may be enabled in accordance with the battery percentage of the electronic device 301 falling below a predetermined threshold (e.g., 30%, 25%, 20%). When operating in the low power mode, certain capabilities may not be enabled on the electronic device 301. For example, the brightness of the display 320 may be decreased if the electronic device 301 is in the low power mode. This may result in the one or more images being displayed with lower luminance values than would be the case while not operating in the low power mode. It should be noted that the electronic device 301 may be in a low power mode based on a battery percentage threshold, but the limited functionality may be similar to that of the electronic device 301 exceeding the power draw threshold described herein. As such, the low power mode may intrinsically maintain power draw from display 320 below the power draw threshold and ensure the safety of the user of the electronic device.

In some examples, baseline pupil sizes for the user of the electronic device, as described in further detail herein, may be different due to the decrease in brightness of the display 320 in response to the electronic device 301 being in a low power mode or when the power draw of the display 320 of the electronic device 301 exceeds the predetermined threshold. In some examples, the baseline pupil sizes may be adjusted in response to the decrease in brightness, in the low power mode or when the power draw of the display 320 of the electronic device 301 exceeds the predetermined threshold. For example, the baseline pupil sizes when the electronic device 301 is not in a limited functionality mode may be set based on a set of optimal conditions (e.g., user health, ambient lighting) being satisfied such that subsequent measurements of the pupil sizes of the one or more eyes of the user may be compared to the baseline pupil sizes to make determinations about that health of the one or more eyes of the user. However, when the electronic device 301 is in a limited functionality mode, the baseline pupil sizes may be different than when the electronic device 301 is not in the limited functionality mode. In some examples, the baseline pupil sizes when the electronic device is in the limited functionality mode may be adjusted based on a scaling factor similar to the decrease in brightness of the display 320. For example, the brightness of the display 320 may be decreased by fifty percent when the electronic device 301 is in a limited functionality mode and in accordance with the decrease in brightness of the display 320, the baseline pupil sizes may scaled up by fifty percent. Additionally, or alternatively, additional baseline pupil sizes may be measured when the electronic device 301 is in a limited functionality mode and when the optimal conditions are satisfied. It should be noted that the baseline pupil sizes increase when the electronic device 301 is in a limited functionality mode because when there is less light exposure to the one or more eyes of the user, the respective pupils increase in size. In some examples, particular determinations may be made when the electronic device 301 is in the low power mode. Because the brightness of all the images may be lower than their values while not in limited functionality mode, the electronic device optionally makes determinations on how the one or more eyes of the user responds to lower light settings. For example, the pupil sizes of the user when viewing the one or more images being displayed at a lower brightness value may have fewer deviations than when the brightness values are normal (e.g., when the electronic device is not operating in the low power mode).

Further, in some examples, sequences of images for eye testing may be identified based on applications installed on the electronic device 301 that include a sequence of images. Additionally or alternatively, applications can be designed to include such sequences of images. For example, when turning the electronic device 301 on or off, or when opening or closing apps, there may be a particular sequence of images showing digital content. In some examples, a sequence of images could be an open sequence for applications (e.g., opening a social media app). In some examples, toggling between light and dark scenes in immersive content (e.g., virtual reality) and/or switching between dark mode and light mode within an application may provide an opportunity to measure pupil sizes of one or more eyes of the user of the electronic device 301. The identified sequence of images from applications installed on the device may be identified because the electronic device 301 may advantageously use the transitions to monitoring ocular behavior. It should be noted that the identified sequence of images need not be curated to monitor ocular behavior, but can be used as the stimuli to opportunistically observe a change in pupil size of the one more eyes of the user of the electronic device 301 (e.g., a pupillary response test).

Therefore, in some examples, while displaying the one or more images using the display 320, the one or more cameras 304 may obtain one or more images of the eyes of a user of the electronic device 301. Depending on the one or more image characteristics of the one or more displayed images, the electronic device 301 may have an expected pupillary response for the one or more cameras 304 to capture. For example, one or more images with high luminance values (e.g., bright images) may correspond to a relatively smaller expected pupil size in comparison to one or more images with low luminance values (e.g., dark images) corresponding to a relatively larger expected pupil size.

FIGS. 3A-3C illustrate the electronic device 301 with a display having varying brightness levels. For example, the electronic device 301 in FIGS. 3A and 3C has a relatively brighter display (e.g., higher luminance values), while the electronic device 301 in FIG. 3B has a relatively darker display (e.g., lower luminance values). Accordingly, the expected pupillary response corresponding to images shown FIGS. 3A and 3C may be smaller pupil sizes while the expected pupillary response corresponding to the image shown in FIG. 3B may be a larger pupil size. In some examples, the one or more images being displayed by the display 320 may transition between displays (e.g., FIG. 3A to FIG. 3B). In some examples, the transition from FIG. 3A to FIG. 3B may be gradual (e.g., over the span of a few images). Additionally, or alternatively, the transition from FIG. 3A to FIG. 3B may be instantaneous (e.g., immediately succeeding images).

It should be noted that although the expected pupil size may be based on a present image, the preceding image(s) may need to be considered. For example, with a gradual transition from a bright image to a dark image, the pupil size of the one or more eyes of the user may increase gradually with the transition of images. However, an instantaneous transition from a bright image to a dark image may not cause an immediate change in pupil size of the one or more eyes of the user. The pupil size of the one or more eyes of the user may increase to the expected pupil size, but there may be delay. In some examples, if there is more than one transition between the one more images, the pupil size of the one or more eyes of the user may be obtained, via the one or more cameras 304, at the beginning of the sequence of images (e.g., before and/or while displaying the first image in the sequence), and after and/or while the final image of the sequence is displayed.

In some examples, the one or more cameras 304 may obtain images of the eye of the user while the electronic device 301 displays the sequence of images. Turning to FIG. 3D, a graph of time versus luminance and/or power draw is shown. Additionally, FIG. 3D illustrates relative pupil sizes based at least on the luminance/power draw of the display of the electronic device 301. It should be noted that the pupil sizes shown in FIG. 3D are relative to one another and correspond to the images shown in FIGS. 3A-3C as an illustrative example. For example, FIG. 3A corresponds to first section 324 of the graph in FIG. 3D, and also corresponds to first eye 328. First section 324 may indicate a relatively high luminance value and power draw from display 320 and correspond to eye 328 with a smaller pupil size than the pupil sizes of eyes 336, 334, and 352. At second section 332, the luminance/power draw of the display may decrease. This decrease may be caused due to a display of a dimmer image than the image in FIG. 3A. In some examples, the decrease in luminance/power draw may be due to the image corresponding to first section 324 exceeding a power draw threshold and causing the electronic device to decrease the brightness (e.g., luminance) of the image. The decrease in luminance/power draw shown at second section 332 corresponds to a slightly larger pupil size shown in second eye 336 compared to eye 328. Further, at third section 340, the luminance/power draw of the display may decrease in comparison to second section 332. This decrease may be due to the electronic device displaying a dimmer image than the image previously displayed (e.g., displayed immediately prior to the image in FIG. 3B). In some examples, the decrease in luminance/power draw may be caused due to the image corresponding to second section 332 exceeding a power draw threshold and causing the electronic device to decrease the brightness (e.g., luminance) of the image. The decrease in luminance/power draw shown at third section 340 corresponds to a slightly larger pupil size shown in third eye 344 compared to eyes 328 and 336.

Additionally or alternatively, a fourth section 348 of the graph may indicate an increase in luminance/power draw of the display 320 of the electronic device 301. This may be caused by an increase in luminance of one or more images shown on the display 320 relative to the luminance of the third section 340. This increase in luminance, shown by fourth section 348, compared to third section 340 corresponds to fourth eye 352. As shown in FIG. 3D, the fourth eye 352 has a pupil size smaller than third eye 344; indicating that the fourth eye 352 may be exposed to brighter light source, or more light in general, relative to third eye 344. As a further example, a fifth section 356 may indicate an increase in luminance/power draw of the display 320 of the electronic device 301. This may be caused by an increase in luminance of one or more images shown on the display 320 relative to the luminance of the fourth section 348. This increase in luminance, shown by fifth section 356, compared to fourth section 348 corresponds to fifth eye 360. In some examples, the electronic device 301 may be used to perform a pupillary light reflex (PLR) test. In some examples, the PLR test may include the display 320 displaying one or more images with luminance values that generally increases and generally decrease in a pattern. For example, the display 320 may display a dim image with an object to focus on (e.g., a dot) with low luminance values to obtain one or more images of pupils of the user. In a subsequent image, the display 320 may display a bright image with the same object to focus on. The one or more cameras 304 may then obtain additional images of the pupils of the user. The dim and bright images may be alternatively displayed while the one or more cameras 304 obtain respective images of the pupils of the user. This process may be repeated to ensure accuracy and precision of the respective images. In some examples, the PLR test may be used to obtain a baseline for the pupil behavior of the user of the electronic device 301. While the PLR testing may be done with minimal environment passthrough, it should be noted that the physical environment that the user of the electronic device 301 is in may also have an effect on the pupil size of the one or more eyes of the user of the electronic device.

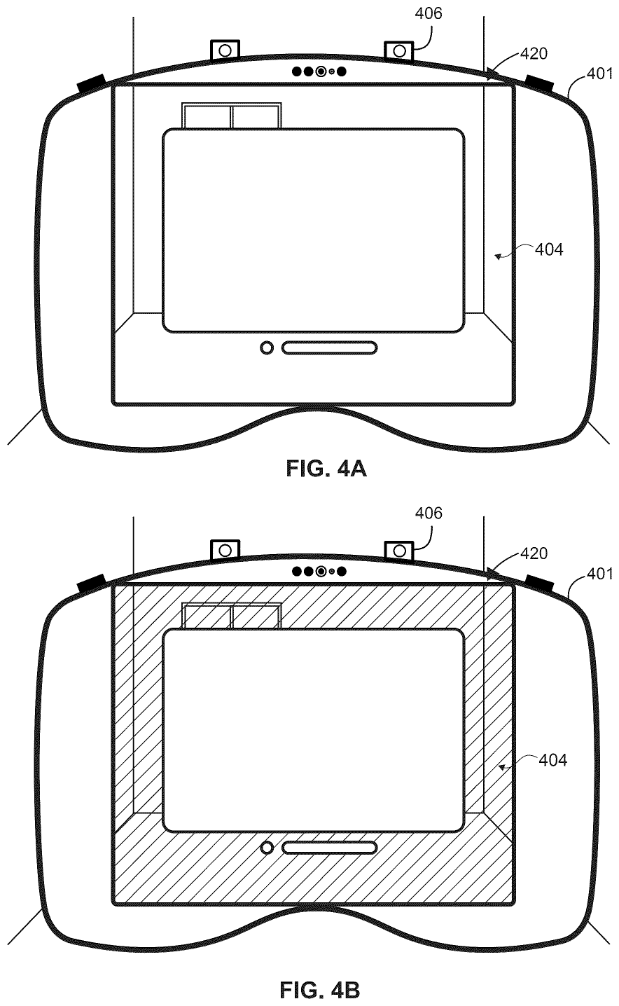

FIGS. 4A and 4B illustrate an electronic device 401 with varying environment passthrough levels. It should be noted that the environment passthrough 404 may be a “real” passthrough. For example, the environment passthrough 404 may include the display 420 presenting the physical surroundings of the electronic device 401 by way of the physical surroundings being visible to the use through a transparent portion of the display. In another example, the environment passthrough 404 may be a “virtual” passthrough. That is, the display 420 may display the passthrough as virtual content in the background of other digital content that the display 420 is displaying. The process described herein relating to the environment passthrough may be optionally applied to either type of environment passthrough mentioned above. As mentioned above, the environment passthrough 404 may affect the pupil size of the one or more eyes of the user of the electronic device 401. In some examples, each eye of the one or more eyes of the user may react different to the environment passthrough 404 and/or the content being displayed on the electronic device 401. It should be noted that the environment passthrough refers to the visibility of the physical environment while the user of the electronic device is viewing digital content. In some examples, a high level of passthrough may allow significant light in (e.g., a bright environment) or allow minimal light in (e.g., a dimly lit environment). In some examples, a low level of passthrough may modify (e.g., limit or increase) an amount of light presented to the user of the electronic device from the physical environment. It should be noted that electronic device 401 may be the same or similar to any electronic device described herein. In some examples, an amount of environment passthrough 404 may be adjusted by the user of electronic device 401. For example, the user of the electronic device 401 may increase or decrease the amount of environment passthrough 404 that the user sees. In some examples, as shown in FIG. 4A, a high amount of environment passthrough 404 may indicate that the user's physical environment that is not blocked by the virtual window may be relatively visible while using the electronic device 401. In some examples, the environment passthrough being visible may change how the electronic device 401 presents one or more images. For example, if the user is an area with a significant amount of sunlight and the environment passthrough 404 of the electronic device 401 is high, then the virtual content being displayed (e.g., the virtual window) may be difficult to see. Accordingly, electronic device 401 may modify one or more image characteristics (e.g., luminance values) associated with the one or more images being displayed on the electronic device 401.

In some examples, the pupil size of the one or more eyes of the user of the electronic device 401 may be dependent on the environment passthrough 404. For example, a bright environment passthrough 404 (e.g., using the electronic device in direct sunlight) may cause the pupil size of the one or more eyes of the user to be smaller than the pupil size would be in a less bright environment. Due to the environment passthrough 404 causing the pupil size of the one or more eyes of the user to be relatively smaller, the images of the one or more eyes of the user, obtained by one or more cameras 406, may include one or more pupil sizes that deviate from an expected size corresponding to the virtual content the electronic device displays. The deviation may be in response to the pupil sizes of the one or more eyes of the user being relatively smaller than usual due to the effect of the environment passthrough 404. In some examples, a dim environment passthrough 404 (e.g., in a room without lights on) may cause the pupil size of the one or more eyes of the user to be larger than the pupil size would be in a brighter environment In some examples, with a high level of environment passthrough 404, transitions from a bright environment to a dim environment, or vice versa, may be exploited to collect data about the behavior of the one or more eyes of the user. The drastic change in brightness may emulate a bright light quickly being shone on the one or more eyes of the user in a traditional pupillary response test. Advantageously, the electronic device 401 may be able to monitor the physiology of the one or more eyes or the user to determine any health condition that may arise over time.

Alternatively, as shown in FIG. 4B, a low amount of environment passthrough may indicate that the user's physical environment is barely visible while using the electronic device 401. This may be because the electronic device is adding a digital effect (e.g., a blur) to the portions of the display presenting the physical environment. Additionally, or alternatively, the user's physical environment may be barely visible due to the physical environment being dark. For example, the user may be in a bright environment and the electronic device may add a digital effect (e.g., a shading) to the portions of the display presenting the physical environment to limit the amount of passthrough from the physical environment. In some examples, the environment passthrough being barely visible may change how the one or more images are displayed on the display of the electronic device 401. For example, if the environment passthrough 404 of the electronic device 401 is low, then the one or more images being displayed may appear more contrasted, and in some examples, appear more crisp. Accordingly, electronic device 401 may modify one or more image characteristics (e.g., luminance values) associated with the one or more images being displayed using the electronic device 401. In some examples, the pupil size of the one or more eyes of the user of the electronic device 401 may be dependent on the environment passthrough 404. For example, a dim environment passthrough 404 may cause the pupil size of the one or more eyes of the user to be larger than the pupil size when the environment passthrough is brighter. Due to the environment passthrough 404 causing the pupil size of the one or more eyes of the user to be relatively larger, the images of the one or more eyes of the user, obtained by one or more cameras 406, may include one or more pupil sizes that deviate from an expected size associated with the one or more images being display on the electronic device. The deviation may be in response to the pupil sizes of the one or more eyes of the user being relatively larger than usual due to the environment.

In some examples, the effect of the environment passthrough 404 on the pupil sizes of the one or more eyes of the user may be corrected by utilizing a pre-measurement algorithm. In some examples, the pre-measurement algorithm may account for the environment passthrough 404 described above before capturing one or more images of the one or more eyes of the user of the electronic device. For example, when the environment passthrough 404 is high, one or more cameras 406 on the electronic device 401 may identify that the environment is bright. As such, the electronic device 401 may then determine that the brightness of the environment, in combination with a high level of environment passthrough 404, may cause the pupil sizes of the one eyes of the user of the electronic device to be smaller than normal. Accordingly, a pre-measurement algorithm may be used to adjust one or more images being displayed on the display of the electronic device 401. Continuing the above example, if the electronic device 401 has a high level, bright, environment passthrough 404, then the one or more images being displayed on the display of the electronic device may have their brightness levels increased. Additionally, or alternatively, if the electronic device 401 has high level, dim, environment passthrough 404, then the display of the electronic device may display the one or more images at relatively lower brightness level. Advantageously, this may reduce the strain on the one or more eyes of the users while also reducing the power draw of the display.

In some examples, the effect of the environment passthrough 404 on the pupil sizes of the one or more eyes of the user may be corrected by utilizing a post-measurement algorithm. In some examples, the post-measurement algorithm may be applied to one or more images of the one or more eyes of the user of the electronic device. For example, the electronic device 401 may be in an environment with dim lighting and have a high level of environment passthrough 404. As such, the pupil sizes of the user of the electronic device may be larger than usual. Accordingly, the post-measurement algorithm may be applied to the measurement of the pupil sizes of the one or more eyes of the user that is determined from the one or more images of the one or more eyes of the user of the electronic device 401. The post-measurement algorithm may include image analysis correction factors that may correct the measurement of the pupil sizes of the one or more eyes of the user. For instance, the image analysis correction factors may enhance the one or more images such that the one or more eyes of the user may be analyzed to measure their respective pupil sizes. It should be noted that the any suitable image analysis methods may be applied to analyze the one or more images of the one or more eyes of the user. In some cases, the image analysis of the one or more sequences of images of the one or more eyes of the user may be utilized to identify an expected pupil size associated with the user of the electronic device, based on one or more images being displayed on the display of the electronic device, environment passthrough 404, or any factors contributing to a change in pupil size.

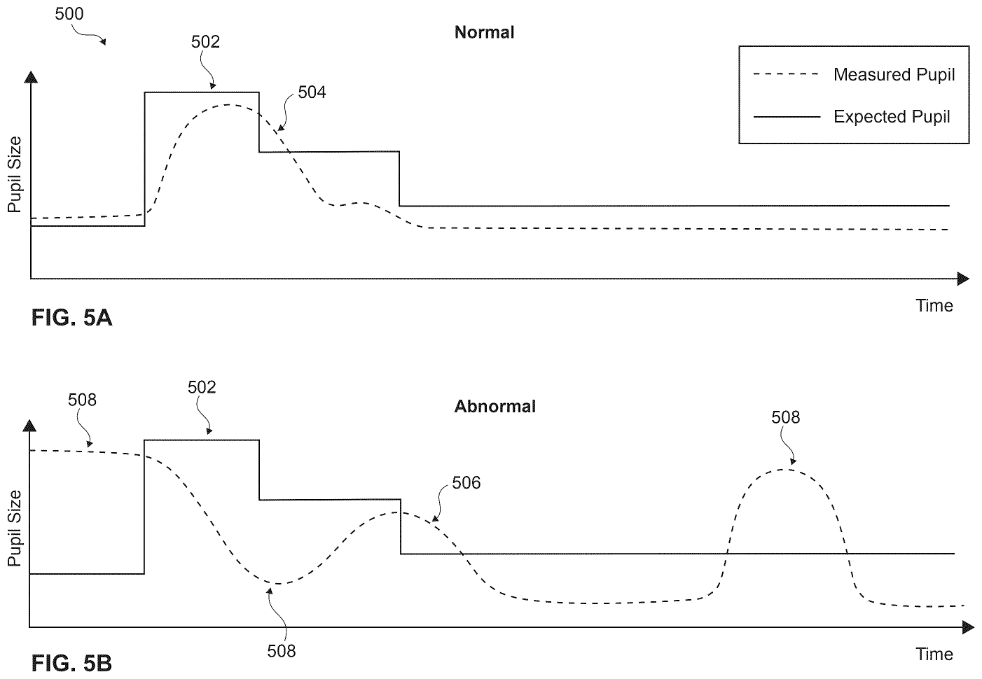

FIGS. 5A-5B illustrate a graph showing a comparison between a measured pupil size and an expected pupil size. In some examples, the expected pupil size 502 may be set when an electronic device is an environment with nominal conditions. For example, when a user of the electronic device is viewing a sequence of images on the electronic device in a room with optimal lighting. The nominal conditions may be predetermined and known by the electronic device. It should be noted that the expected pupil size may be measured when the sequence of images being displayed on the electronic device include known luminance values. As such, the expected pupil size may correspond to the known luminance values. In some examples, the expected pupil size may not be measured based on a single sequence of images. As mentioned above, the expected pupil size may correspond to the known luminance values of a sequence of images, but a single sequence of images may not yield sufficient measurements. Accordingly, additional sequences of images, also with known luminance values, may be displayed to measure the expected pupil size of the user of the electronic device. Additionally or alternatively, the expected pupil size may be set using a machine learning model. For example, a pupil size machine learning model may be trained using training data that correlates historical luminance values to historical pupil sizes. In some examples, the training data may include data from the user of the electronic device. In some examples, the training data may include data from users of other electronic devices. In some examples, the pupil size machine learning model may include one or more constraints. For example, the one or more constraints may include, but are not limited to, age of a user, an emotional state of a user, and/or an environment of a user. In some examples, the one or more constraints may be manually input by the user. In some examples, the user inputs may include a user's mood, an amount of time spent sleeping over a time interval, one or more preexisting health conditions, or the like. In some examples, the electronic device may have access to data stored in one or more health related applications on the electronic device. As such, the electronic device may use the stored health data from the one or more health related applications. In some examples, the stored health data may include data relating to the user's emotional state, mood, sleep patterns, diet, or things of the like.

In some examples, the pupil size machine learning model may be iterative. For example, the training data may be input into an iterative algorithm that has one or more parameters that are optimized through multiple iterations (e.g., different inputs). In some examples, the one or more parameters may be associated with the one or more constraints mentioned above. It should be noted that the expected pupil size, independent of the method in which it is set, may be updated periodically (e.g., every day, every week, every month) through the same, or different, method that it was initially set with.

In FIG. 5A, normal pupil size graph 500 is shown. The normal pupil size graph 500 provides a graphical representation of the relationship between expected pupil size 502 and measured pupil size 504. As shown, the measured pupil size 504 does not align with the expected pupil size precisely, but generally follows the same behavior. In some examples, the measured pupil size 504 may be a response curve. The measured pupil size 504 may also be dose dependent response curve, where the dose refers to light from any light source as described herein. As mentioned above, the expected pupil size 502 may be associated with luminance values being displayed on the display of the electronic device. In some examples, the expected pupil size 502 may be the expected pupil size in ideal conditions for a user. However, in most cases, the user of the electronic device is in non-ideal conditions, causing the measured pupil size 504 to differ slightly from the expected pupil size 502. If the measured pupil size 504 differs from the expected pupil size within a threshold amount, then the measured pupil size 504 may be classified as “normal.”

In some examples, the measured pupil size 506 may differ more than a threshold amount from the expected pupil size 502. For example, in FIG. 5B, deviations 508 may indicate that pupils of the user of the electronic device are not having the intended response to the one or more images being displayed. The discrepancy may be caused by an issue with the one or more eyes of the user of the electronic device. In some examples, the discrepancy causing the deviations 508 may be the one or more images being displayed, or an error in the display itself. For example, the one or more images being displayed may have a lower luminance value than the expected, causing the pupil sizes of the user of the electronic device to be larger than expected. In some examples, the one or more images being displayed may be include one or more incorrect luminance values. As such, there may be a discrepancy between the expected pupil size 502 and measured pupil size 506. In some examples, the discrepancy between the expected pupil size 502 and the measured pupil size 506 may be attributed to a physiological change in the one or more eyes of the user of the electronic device. For example, the one or more eyes may change over time due to age and/or emotional state. Based on the deviations, one or more indications may be generated and presented to the user of the electronic device. The deviations may exceed a predetermined pupil size threshold. The one or more indications may include visual indications, audio indications, haptic indications, one or more notifications to other devices, or things of the like. The one or more indications may provide information to at least the user of the electronic device regarding the discrepancy between the measured pupil size and the expected pupil size. The one or more indications may be displayed as a pop-up window on the display of the electronic device. In one or more examples, the one or more indications may prompt the user of the electronic device to initiate an active session of pupil reactivity that may allow for more accurate measurements. For example, the active session of pupil reactivity may include displaying specific instructions and images to measured one or more changes in pupil size of the one or more eyes of the user of the electronic device. In some examples, the one or more indications may be sent to a medical professional. For example, the one or more indications may indicate potential eye conditions, and that may be communicated to a medical professional so that the medical professional may provide one or more recommendations to address the potential eye ailment. However, in some examples, the measured pupil size 506 may be out of phase with the expected pupil size 502. For example, the expected pupil size 502 may have a small pupil size at a first time and then a large pupil size at a second time. However, the measured pupil size may indicate a large pupil size at the first time and a small pupil size at the second time. This measurement may serve as a metric for a pupillary response time of the one or more eyes of the user of the electronic device. For example, a time difference between a luminance/power draw peak of the measured pupil size 506 and a corresponding peak of the expected pupil size 502 may be directly proportional to the pupillary response time associated with the one or more eyes of the user of the electronic device. For example, a luminance/power draw peak for both the measured pupil size 506 and the expected pupil size 502 may be substantially located at a same time mark. As such, the pupillary response time may low. In another example, a luminance/power draw peak for both the measured pupil size 506 and the expected pupil size 502 may be located at significantly different time marks. The significant difference in time marks may indicate a high pupillary response time. It should be noted that deviations 508 may be monitored over a period of time (e.g., 1 day, 1 week, 1 month) prior to generating the one or more indications. The monitoring of the deviations 508 may ensure that the deviations are accurate and precise.

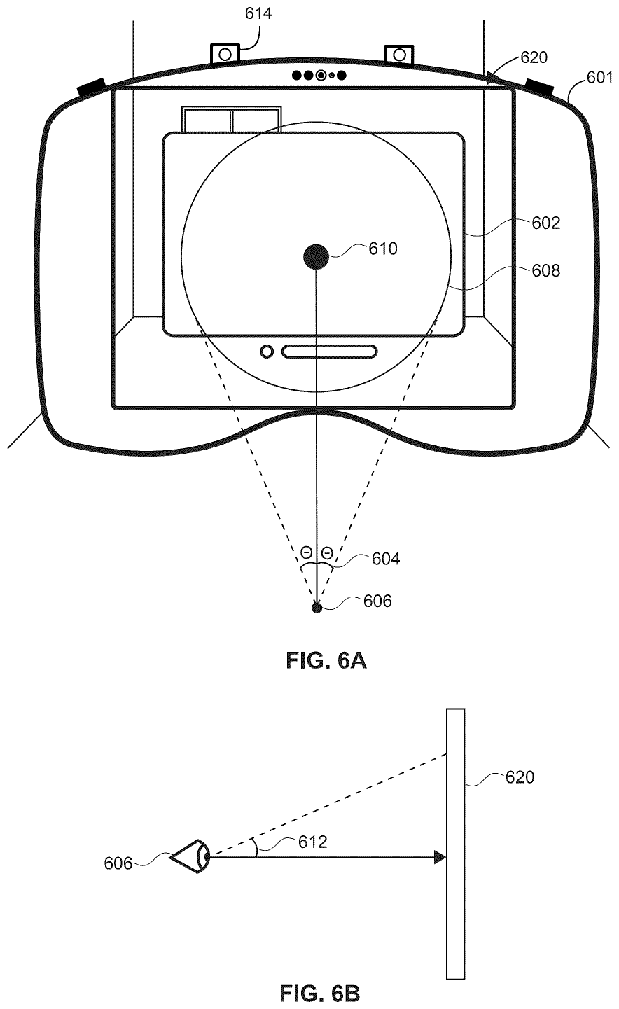

In FIG. 6A, an exemplary display 620 of an electronic device 601 (e.g., electronic device 201, 301, 401) is shown. In some examples, the display 620 may include a virtual window 602. For example, virtual window 602 may include one or more images being displayed to the user of the electronic device. In some examples, the one or more images may be displayed in background of the virtual window 602. For example, the one or more images may be a video being displayed while the virtual window 602 may include a pop-up associated with the video being displayed or a separate application running on the electronic device. In some cases, the one or more images, either being displayed in the virtual window 602 or at another location, may have known luminance values as mentioned above. However, to ensure that accurate pupil size measurements are made, the display may display the one or more images corresponding to the known luminance within a threshold angular field of view. For example, as shown in FIG. 6A, the horizontal threshold angle 604 is shown relative to the display 620 and the eye 606. The horizontal threshold angle 604 to the display 620 has a maximum field of view as shown by perimeter 608. In some examples, the horizontal threshold angle may be set by an amount of pupil that is in view of the one or more cameras 614. When the one or more cameras 614 obtain one or more images of the one or more eyes of the user the full respective pupil of the one or more eyes of the user must be visible to measure one or more measured pupil sizes. The horizontal threshold angle 604 may be set based on a maximum angle that the one or more eyes of the user may look off a central, vertical, axis while having the full respective pupil of the one or more eyes of the user visible. A vertical threshold angle 612, described further below, may be set based on a maximum angle that the one or more eyes of the user may look off a central, horizontal, axis while having the full respective pupil of the one or more eyes of the user visible.

In some examples the one or more images, with known luminance values, do not have to fall within the perimeter 608. However, the display displaying the one or more images outside of the perimeter 608 may require corrections to the measured pupil sizes. Further, knowing how far outside of the perimeter 608 that the display is displaying the one or more images may account for the effective dosage of light hitting the one or more eyes' photoreceptors and normalize measurements taken outside of perimeter 608. Continuing the above example, the perimeter 608 may include a gaze point 610. The gaze point 610 may be direct line-of-sight from the center of the pupil of eye 606 to the display 620 (e.g., the center of perimeter 608). In some examples, the gaze point 610 may also indicate that all of a respective pupil of the one or more eyes is visible by the one or more cameras 614. Ultimately, having the gaze point of the one or more eyes of the user of the electronic device within the perimeter 608 may indicate that the respective pupils of the one or more eyes of the user satisfy the angular thresholds. If the gaze point falls outside of the perimeter 608, then the one or more cameras 614 may forgo obtaining one or more images of the one or more eyes of the user of the electronic device 601. It should be noted that the FIG. 6A is exemplary for one eye of the user of the electronic device. Any pupil measurement methods disclosed herein may be performed for both eyes of the user of the electronic device. In some examples, perimeter 608 for a first eye, may overlap with a perimeter of a second eye. As such, the one or more images being displayed on the display 620 may be within the horizontal angular threshold of the one or more eyes of the user of the electronic device.

Additionally, there may be a vertical angular threshold for the one or more eyes of the user of the electronic device, as shown by FIG. 6B. The vertical threshold angle 612 may be the same value as the horizontal threshold angle 604. In some examples, the vertical threshold angle 612 may be different that the horizontal threshold angle 604. In some examples, the vertical threshold angle may be constrained by the physical parameters of the display 620 For example, relative to the position of the eye 606, the vertical threshold angle 604 may allow for a vertical gaze range that extends past the display 620. As such, in some examples, the horizontal threshold angle 604 may have more significance in outlining the perimeter 608, signifying which portion of the display 620 that the one or more images being displayed would yield accurate pupil size measurements.

Movement of the electronic device 601 may be caused by the user of the electronic device. For example, the user may be walking, jogging, running, or the like, and that may cause the electronic device 601 to move from a stationary position. To correct for the movement of the electronic device 601, the display 620 may display a notification to instruct the user of the electronic device 601 to adjust the electronic device 601, on the head of the user, in order to ensure accurate eye tracking by the one or more cameras 614. In some examples, the movement of the electronic device 601 may cause difficulty in obtaining the one or more images of the eyes of the user of the electronic device 601. It should be noted that the threshold angles are measured relative to the eye 606. However, one or more cameras 614 disposed on the vertical plane of the electronic device 601 are configured to obtain the pupil size measurements.

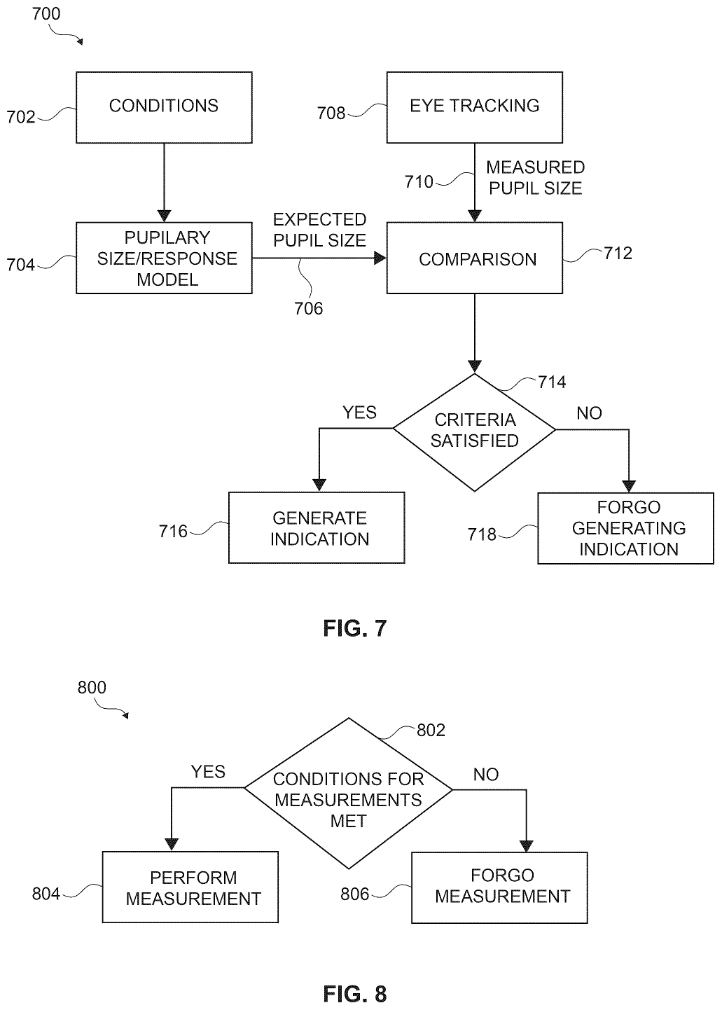

FIG. 7 is a flow chart of an example method 700 of generating an indication of a deviation from the expected pupillary response in accordance with one or examples in the disclosure. In some examples, an electronic device (e.g., 201, 301, 401, 601) performs method 700 as described herein. Optionally, one or more steps of the method 700 are programmed in instructions stored using non-transitory computer readable storage media.

At 702, the electronic device may obtain and/or measure one or more conditions associated with a user of the electronic device and the electronic device itself. In some examples, the one or more conditions may include, but are not limited to, motion of the electronic device, temperature of the electronic device, eye movement, gaze direction, luminance levels, age of the user, mood of the user, or things of the like. As described herein, the one or more conditions may be input into a pupil size model as one or more constraints. In some examples, the one or more conditions may be required to satisfy one or more additional conditions to perform the rest of the method.

At 704, the electronic device may utilize a pupil size model to generate an expected pupil size 706, as described herein. In some examples, utilizing the pupil size model may include training the model with historical pupil response data as described above. In some examples, as described herein, the pupil size model may be trained iteratively to ensure accuracy and precision of the model.

At 708, one or more cameras of the electronic device may perform eye tracking on the user of the electronic device. As described herein, the eye tracking may be performed to measure a measured pupil size 710 of one or more eyes of the user while the user is viewing one or more images on the display of the electronic device. In some examples, as described herein, the eye tracking may be used to measure a baseline pupil size when conditions satisfy one or more criteria. Measuring the pupil size of the one or more eyes of the user may depend on a gaze direction of the one or more eyes of the user. The gaze direction being outside of a gaze perimeter may cause the one or more cameras forgo obtaining one or more images of the one or more eyes of the user. In some examples, the one or more cameras may selectively track one eye to preserve power. In some examples, the electronic device may perform different corrections for each eye of the one or more eyes of the user.

At 712, the electronic device may compare the measured pupil size 710 to the expected pupil size 706, as described herein. In some examples, as described herein, the comparison may include identifying one or more discrepancies between the measured pupil size and the expected pupil size. The one or more discrepancies may indicate a deviation from the expected pupillary response, as described herein. In some examples, the deviation may be attributed to the physiology of the user of the electronic device, the display of the electronic device, and/or the one or more cameras of the electronic device. For example, the measured pupil size may be similar the expected pupil size but is offset based on time. That is, the curve representing measured pupil size may be shifted to the right (e.g., an increase in time) relative to the expected pupil size. This may indicate that the deviation is caused by the electronic device and not the physiology of the eye of the user. In another example, the curve representing measured pupil size show random behavior (e.g., FIG. 5B) relative to the expected pupil size. This may indicate that the deviation is caused by the physiology of the eye of the user since the measured pupil size has no correlation to the expected pupil size.

At 714, the electronic device may determine whether one or more criteria are satisfied. As described herein, the one or more criteria may be satisfied when a deviation between the expected pupil size and the measured pupil size exceed a threshold. For example, one or more criteria may be satisfied if percent difference exceeds a predetermined threshold (e.g., 15%, 20%). If the one or more criteria (e.g., the deviation exceeds a threshold pupil size) are satisfied, then the electronic device may generate an indication 716. If the one or more criteria are not satisfied, then the electronic device may forgo generating the indication 718. As described herein, the generated indication may include visual indications, audio indications, haptic indications, one or more notifications to other devices, or things of the like. In some examples, the generated indication may include one or more signs of ocular diseases and/or disorders (e.g., afferent pupillary defect (APD), Horner's syndrome, Adie's tonic pupil, Argyll Robertson pupil).

FIG. 8 is a flow diagram illustrating an example process for determining whether to opportunistically measure pupil sizes of one or more eyes of a user of an electronic device (e.g., 201, 301, 401, 601) is shown. At 802, the electronic device may determine whether one or more conditions are met to perform one or more measurements. As described herein, in some examples, the one or more conditions may include motion of the electronic device, temperature of the electronic device, eye movement, gaze direction, luminance levels, or things of the like. If the one or more conditions are satisfied, then the electronic device proceeds with performing one or more pupil size measurements of the one or more eyes of the user of the electronic device at 804. If the one or more conditions are not satisfied, then electronic device will forgo performing one or more pupil size measurements of the one or more eyes of the user of the electronic device at 806.

Therefore, according to the above, some examples of the disclosure are directed to a method comprising at an electronic device in communication with one or more displays and one or more input devices: displaying, via the one or more displays, one or more images; while displaying the one or more images, detecting, via the one or more input devices, one or more first sizes of a pupil of a user of the electronic device; comparing the one or more first sizes of the pupil detected, while displaying the one or more images, with one or more second sizes of the pupil of the user of the electronic device, the or more images; and in accordance with a determination that one or more criteria are satisfied, including a criterion that is satisfied when the one or more first sizes of the pupil deviate from the one or more second sizes of the pupil by one or more pupil size thresholds, generate an indication of a deviation from an expected pupillary response.

Additionally or alternatively, in some examples, the method further comprises determining one or more luminance characteristics of the one or more images. Additionally or alternatively, in some examples, the method further comprises determining a power draw of the one or more displays while displaying the one or images. Additionally or alternatively, in some examples, the one or more images includes a sequence of images, and wherein one or more luminance characteristics changes as the sequence progresses. Additionally or alternatively, in some examples, the sequence of images includes a threshold increase in the one or more luminance characteristics corresponding to a transition from less than a first threshold luminance to greater than a second threshold luminance, the second threshold luminance greater than the first threshold luminance, as the sequence progresses. Additionally or alternatively, in some examples, the sequence of images includes a threshold decrease in the one or more luminance characteristics corresponding to a transition from greater than a first threshold luminance to less than a second threshold luminance, the second threshold luminance less than the first threshold luminance, as the sequence progresses. Additionally or alternatively, in some examples, the expected pupillary response to the one or more images is based on a prior pupillary response to the one or more images. Additionally or alternatively, in some examples, the expected pupillary response to the one or more images is based on a pupillary response model.

Additionally or alternatively, in some examples, the pupillary response model outputs the one or more second sizes based on one or more inputs including at least one of an age of the user of the electronic device, a mood of the user of the electronic device, one or more luminance characteristics of the one or images, power consumption of the one or more displays used to display the one or more images, or a gaze direction relative to the one or more images. Additionally or alternatively, in some examples, the method further comprises determining a gaze direction of the user of the electronic device, wherein detecting the one or more first sizes of a pupil of a user of the electronic device occurs while the gaze direction is within a threshold angular field of view. Additionally or alternatively, in some examples, in accordance with a determination that the one or more criteria are not satisfied, forgo generating the indication of a deviation from the expected pupillary response. Additionally or alternatively, in some examples, detecting the one or more first sizes of the pupil comprises performing a series of detections over predetermined interval of time, and wherein the predetermined interval of time is determined by a frequency that the one or more images are displayed. Additionally or alternatively, in some examples, the one or more criteria include a criterion that is satisfied when the one or more first sizes of the pupil deviate from the one or more second sizes of the pupil by the one or more pupil size thresholds for a threshold number of measurements over a threshold period of time.

Additionally or alternatively, in some examples, detecting the one or more first sizes of the pupil of the user of the electronic device occurs while the electronic device movement is less than a threshold. Additionally or alternatively, in some examples, detecting the one or more first sizes of the pupil of the user of the electronic device occurs based on a thermal condition or power state of the electronic device. Additionally or alternatively, in some examples, detecting the one or more first sizes of the pupil of the user of the electronic device occurs after a threshold period of time elapsed since a prior detection of the one or more first sizes of the pupil of the user and/or a prior comparison of the one or more first sizes of the pupil with one or more second sizes of the pupil. Additionally or alternatively, in some examples, comparing the one or more first sizes of the pupil with one or more second sizes of the pupil occurs when the one or more images correspond to a predetermined sequence of images.

Some examples of the disclosure are directed to a head-mounted device. The head-mounted device includes, one or more output devices including one or more displays, one or more eye tracking sensors, and one or more processors. The one or more processors are configured to display, using the one or more displays, a sequence of images that include a variation in luminance. In some examples, the head-mounted device detects, using the one or more eye tracking sensors, one or more changes of a size of a pupil of a user of the head-mounted device while displaying the sequence of images, and compares the one or more changes of the size of the pupil of the user of the head-mounted device to one or more expected changes of the size of the pupil of the user of the head-mounted device estimated based on the variation in luminance of the sequence of images. In some examples, in accordance with a determination of a deviation indicated by the one or more changes of the size of the pupil of the user from the one or more expected changes of the size, the head-mounted device generates, using the one or more output devices, an indication of the deviation.

Some examples of the disclosure are directed to a first electronic device, comprising one or more processors, memory, and means for performing any of the above methods.

Some examples of the disclosure are directed to a non-transitory computer readable storage medium storing one or more programs comprising means for performing any of the above methods.

The present disclosure contemplates that in some examples, the data utilized may include personal information data that uniquely identifies or can be used to contact or locate a specific person. Such personal information data can include demographic data, content consumption activity, location-based data, telephone numbers, email addresses, twitter ID's, home addresses, data or records relating to a user's health or level of fitness (e.g., vital signs measurements, medication information, exercise information), date of birth, or any other identifying or personal information. Specifically, as described herein, one aspect of the present disclosure is tracking a user's biometric data.

The present disclosure recognizes that the use of such personal information data, in the present technology, can be used to the benefit of users. For example, personal information data may be used to display suggested text that changes based on changes in a user's biometric data. For example, the suggested text is updated based on changes to the user's age, height, weight, and/or health history.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates examples in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select not to enable recording of personal information data in a specific application (e.g., first application and/or second application). In addition to providing “opt in” and “opt out” options, the present disclosure contemplates providing notifications relating to the access or use of personal information. For instance, a user may be notified upon initiating collection that their personal information data will be accessed and then reminded again just before personal information data is accessed by the device(s).

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

The foregoing description, for purpose of explanation, has been described with reference to specific examples. However, the illustrative discussions above are not intended to be exhaustive or to limit the disclosure to the precise forms disclosed. Many modifications and variations are possible in view of the above teachings. The examples were chosen and described in order to best explain the principles of the disclosure and its practical applications, to thereby enable others skilled in the art to best use the disclosure and various described examples with various modifications as are suited to the particular use contemplated.

The foregoing description, for purpose of explanation, has been described with reference to specific examples. However, the illustrative discussions above are not intended to be exhaustive or to limit the disclosure to the precise forms disclosed. Many modifications and variations are possible in view of the above teachings. The examples were chosen and described in order to best explain the principles of the disclosure and its practical applications, to thereby enable others skilled in the art to best use the disclosure and various described examples with various modifications as are suited to the particular use contemplated.

What is claimed is:

1. An electronic device in communication with one or more displays and one or more input devices, the electronic device, comprising:
one or more processors;
memory; and
one or more programs, wherein the one or more programs are stored in the memory and configured to be executed by the one or more processors, the one or more programs including instructions for:
displaying, via the one or more displays, one or more images;
while displaying the one or more images, detecting, via the one or more input devices, one or more first sizes of a pupil of a user of the electronic device;
comparing the one or more first sizes of the pupil detected, while displaying the one or more images, with one or more second sizes of the pupil of the user of the electronic device, the one or more images; and
in accordance with a determination that one or more criteria are satisfied, including a criterion that is satisfied when the one or more first sizes of the pupil deviate from the one or more second sizes of the pupil by one or more pupil size thresholds, generate an indication of a deviation from an expected pupillary response.

2. The electronic device of claim 1, wherein the one or more programs further include instructions for:
determining a power draw of the one or more displays while displaying the one or images.

3. The electronic device of claim 1, wherein the one or more images includes a sequence of images, and wherein one or more luminance characteristics changes as the sequence progresses.

4. The electronic device of claim 3, wherein the sequence of images includes a threshold increase in the one or more luminance characteristics corresponding to a transition from less than a first threshold luminance to greater than a second threshold luminance, the second threshold luminance greater than the first threshold luminance, as the sequence progresses.

5. The electronic device of claim 3, wherein the sequence of images includes a threshold decrease in the one or more luminance characteristics corresponding to a transition from greater than a first threshold luminance to less than a second threshold luminance, the second threshold luminance less than the first threshold luminance, as the sequence progresses.

6. The electronic device of claim 1, the expected pupillary response to the one or more images is based on a pupillary response model or a priori pupillary response.

7. The electronic device of claim 6, wherein the pupillary response model outputs the one or more second sizes based on one or more inputs including at least one of an age of the user of the electronic device, a mood of the user of the electronic device, one or more luminance characteristics of the one or images, power consumption of the one or more displays used to display the one or more images, or a gaze direction relative to the one or more images.

8. The electronic device of claim 1, wherein the one or more programs further include instructions for:
determining a gaze direction of the user of the electronic device, wherein detecting the one or more first sizes of a pupil of a user of the electronic device occurs while the gaze direction is within a threshold angular field of view.

9. The electronic device of claim 1, in accordance with a determination that the one or more criteria are not satisfied, forgo generating the indication of a deviation from the expected pupillary response.

10. The electronic device of claim 1, wherein detecting the one or more first sizes of the pupil comprises performing a series of detections over predetermined interval of time, and wherein the predetermined interval of time is determined by a frequency that the one or more images are displayed.

11. The electronic device of claim 1, wherein the one or more criteria include a criterion that is satisfied when the one or more first sizes of the pupil deviate from the one or more second sizes of the pupil by the one or more pupil size thresholds for a threshold number of measurements over a threshold period of time.

12. The electronic device of claim 1, wherein detecting the one or more first sizes of the pupil of the user of the electronic device occurs:
after a threshold period of time elapsed since a prior detection of the one or more first sizes of the pupil of the user and/or a prior comparison of the one or more first sizes of the pupil with one or more second sizes of the pupil;
while movement of the electronic device is less than a threshold; and/or
based on a thermal condition or power state of the electronic device.

13. A method comprising:
at an electronic device in communication with one or more displays and one or more input devices:
displaying, via the one or more displays, one or more images;
while displaying the one or more images, detecting, via the one or more input devices, one or more first sizes of a pupil of a user of the electronic device;
comparing the one or more first sizes of the pupil detected, while displaying the one or more images, with one or more second sizes of the pupil of the user of the electronic device, the one or more images; and
in accordance with a determination that one or more criteria are satisfied, including a criterion that is satisfied when the one or more first sizes of the pupil deviate from the one or more second sizes of the pupil by one or more pupil size thresholds, generate an indication of a deviation from an expected pupillary response.

14. The method of claim 13, further comprising:
determining a power draw of the one or more displays while displaying the one or images.

15. The method of claim 13, wherein the one or more images includes a sequence of images, and wherein one or more luminance characteristics changes as the sequence progresses.

16. The method of claim 13, wherein a pupillary response model outputs the one or more second sizes based on one or more inputs including at least one of an age of the user of the electronic device, a mood of the user of the electronic device, one or more luminance characteristics of the one or images, power consumption of the one or more displays used to display the one or more images, or a gaze direction relative to the one or more images.

17. The method of claim 13, further comprising: determining a gaze direction of the user of the electronic device, wherein detecting the one or more first sizes of a pupil of a user of the electronic device occurs while the gaze direction is within a threshold angular field of view.

18. The method of claim 13, wherein the one or more criteria include a criterion that is satisfied when the one or more first sizes of the pupil deviate from the one or more second sizes of the pupil by the one or more pupil size thresholds for a threshold number of measurements over a threshold period of time.

19. The method of claim 13, wherein detecting the one or more first sizes of the pupil of the user of the electronic device occurs after a threshold period of time elapsed since a prior detection of the one or more first sizes of the pupil of the user and/or a prior comparison of the one or more first sizes of the pupil with one or more second sizes of the pupil;
while movement of the electronic device is less than a threshold; and/or
based on a thermal condition or power state of the electronic device.

20. A head-mounted device, comprising:
one or more output devices including one or more displays;
one or more eye tracking sensors; and
one or more processors configured to:
display, using the one or more displays, a sequence of images that include a variation in luminance;
detect, using the one or more eye tracking sensors, one or more changes of a size of a pupil of a user of the head-mounted device while displaying the sequence of images;
compare the one or more changes of the size of the pupil of the user of the head-mounted device to one or more expected changes of the size of the pupil of the user of the head-mounted device estimated based on the variation in luminance of the sequence of images; and
in accordance with a determination of a deviation indicated by the one or more changes of the size of the pupil of the user from the one or more expected changes of the size, generate, using the one or more output devices, an indication of the deviation.
